SEQUENCE LISTING (1 sequence)

SEQ ID NO: 1 - protein, 360 aa, Artificial Sequence
  CTPR10 protein
GAMGSAEAWYNLGNAYYKQGDYDEAIEYYQKALELDPRSAEAWYNLGNAYYKQGDYDEAIEYYQKALELDPRSAEAWYNLGNAYYKQGDYDEAIEYYQKALELDPRSAEAWYNLGNAYYKQGDYDEAIEYYQKALELDPRSAEAWYNLGNAYYKQGDYDEAIEYYQKALELDPRSAEAWYNLGNAYYKQGDYDEAIEYYQKALELDPRSAEAWYNLGNAYYKQGDYDEAIEYYQKALELDPRSAEAWYNLGNAYYKQGDYDEAIEYYQKALELDPRSAEAWYNLGNAYYKQGDYDEAIEYYQKALELDPRSAEAWYNLGNAYYKQGDYDEAIEYYQKALELDPRSAEAKQNLGNAKQKQG

(19) United States
(12) Patent Application Publication
(10) Pub. No.: US 2024/0052175 A1
(43) Pub. Date: Feb. 15, 2024

(54) PROTEIN-BASED COMPOSITION FOR ADDITIVE MANUFACTURING

(71) Applicants: University of Washington, Seattle, WA (US); Universidad del Pais Vasco/Euskal Herriko Unibersitatea, Bizkaia (ES)

(72) Inventors: Alshakim Nelson, Seattle, WA (US); Eva Sánchez Rexach, Bizkaia (ES); Haritz Sardon, Bizkaia (ES)

(73) Assignees: University of Washington, Seattle, WA (US); Universidad del Pais Vasco/Euskal Herriko Unibersitatea, Bizkaia (ES)

(21) Appl. No.: 18/268,761

(22) PCT Filed: Dec. 21, 2021
(86) PCT No.: PCT/US2021/064530

§ 371 Date: Jun. 21, 2023

Related U.S. Application Data
(60) Provisional application No. 63/129,959, filed on Dec. 23, 2020.

(51) Int. Cl. C09D 4/00; C09D 189/00; A61L 31/04; B33Y 70/00

(52) U.S. Cl. CPC C09D 4/00; C09D 189/00; A61L 31/047; A61L 31/048; B33Y 70/00; B33Y 10/00

(57) ABSTRACT

Disclosed herein are compositions comprising globular proteins and diacrylate-containing compounds that can react in-situ to provide polymerized networks that can be formed into objects using additive manufacturing techniques, such as printing techniques that use vat photopolymerization. Objects printed using the disclosed compositions also are described, with embodiments of such objects exhibiting shape recovery behavior. Also disclosed are methods of making and using the disclosed compositions.

### CROSS REFERENCE TO RELATED APPLICATION

This application claims the benefit of and priority to the earlier filing date of U.S. Provisional Application No. 63/129,959 filed Dec. 23, 2020, which is incorporated by reference herein in its entirety.

### FIELD

The present disclosure is directed to protein-based composition embodiments and methods of making and using the same, particularly to print objects that exhibit improved mechanical properties and exhibit shape recovery behavior.

### BACKGROUND

Bio-based plastics that can supplant petroleum-derived materials are desired to meet the future demands of sustainability in the life cycle of plastic materials. While there are significant efforts to develop protein-based plastic materials for commercial use, their application is limited by poor processability and limitations in mechanical performance.

Additive manufacturing, commonly known as 3D printing, has drawn tremendous attention as a versatile platform for the on-demand fabrication of functional objects with complex architectures. The advent of 3D printing techniques, including direct write, ink jet printing, and vat photopolymerization, has revolutionized manufacturing. These techniques enable the iterative design of complex shapes without the need for additional tooling, operator expertise, or expensive molds. Stereolithography (SLA) is one type of vat photopolymerization where a laser is scanned to selectively cure a photocurable resin to form a structure. Unlike some other types of 3D printing, SLA provides the ability to achieve micron scale features resulting in high accuracy parts. While capable of producing a structure having high resolution 3D geometry, SLA is greatly limited by the lack of available compositions that are compatible with the technique.

There exists a need in the art for a composition that can be used with additive manufacturing techniques to print objects that are biocompatible, can exhibit shape recovery behavior, and also exhibit desirable mechanical properties.

### SUMMARY

Disclosed herein are embodiments of a composition, comprising: a non-acrylated globular protein; a diacrylate-containing compound; a photoinitiating component; and a solvent. Additional features of such composition embodiments are described herein.

Also disclosed herein are embodiments of a printed object, comprising a polymerized network comprising a first globular protein molecule that is covalently bound to a saturated form of a first diacrylate-containing compound, wherein the protein is directly covalently bound to a carbon atom of the saturated form of the first diacrylate-containing compound via a non-acrylated functional group of the protein; and wherein the saturated form of the diacrylate-containing compound is further directly covalently bound to a saturated form of a second diacrylate-containing compound, a second globular protein molecule, or a combination thereof. Additional features of such printed object embodiments are described herein.

Also disclosed herein are embodiments of a method, comprising printing an object using a composition comprising (i) a non-acrylated globular protein; (ii) a diacrylate-containing compound; (iii) a photoinitiating component; and (iv) a solvent, wherein the printing is carried out using an additive manufacturing device that uses an energy source to promote vat photopolymerization. Additional features of such method embodiments are described herein.

The foregoing and other objects and features of the present disclosure will become more apparent from the following detailed description, which proceeds with reference to the accompanying figures.

### BRIEF DESCRIPTION OF THE DRAWINGS

FIG. 1 is a schematic illustration showing compression of printed objects made using a composition embodiment described herein, followed by shape recovery after exposure to heat (top) or exposure to water (bottom).

FIG. 2 is a schematic illustration showing an embodiment of an SLA 3D printing and shape recovery process using composition and method embodiments disclosed herein, wherein two different objects—a “W-shaped” rod and a sphere—were 3D printed as hydrogel objects and then dried to afford the corresponding 3D bioplastic objects; the printed objects were then plastically deformed by compression (sphere) or elongation (“W-shaped” rod) to afford metastable states of the objects, and then exposed to water (sphere) or heat (“W-shaped” rod) to recover their original shape.

FIG. 3 is a schematic illustration showing mixing of composition components according to an embodiment of the present disclosure, along with a description of chemical reactions that occur between the components during the method.

FIGS. 4A and 4B illustrate the isotropic de-swelling of a printed object according to the present disclosure and showing that the bioplastic object (FIG. 4B) retained the same geometry and shape as the printed hydrogel (FIG. 4A) but with 30% smaller dimensions after drying.

FIGS. 5A and 5B are graphs showing results confirming functionalization of amines of a protein compound (BSA) with acrylate groups of a diacrylate-containing compound (PEG-DA), wherein FIG. 5A is a graph of absorbance as a function of wavelength showing that 78% of the lysine residues in the protein reacted with the diacrylate-containing compound and FIG. 5B is a graph of absorbance as a function of wavenumber confirming that the presence of secondary amines formed after lysine residues of the protein reacted with acrylate groups of a diacrylate-containing compound via an aza-Michael addition.

FIGS. 6A and 6B show results obtained from characterizing two different composition embodiments using rheometry to determine the rate of photocure (FIG. 6A), and viscosity (FIG. 6B) for each of the compositions, and further comparing these composition embodiments with (i) a composition comprising only a globular protein (“BSA”) and (ii) a commercial SLA resin (“Formlabs Clear Resin”).

FIGS. 7A and 7B show rheology (FIG. 7A) and viscosity (FIG. 7B) results obtained from evaluating different composition embodiments as compared to a commercial resin (“Formlabs clear resin”) and a composition comprising only a globular protein at 30 wt % (“BSA”) with no diacrylate-containing compound; the different composition embodiments included 30 wt % BSA with (i) 3 wt % poly(ethylene glycol) diacrylate (“PEG-DA”), (ii) 5 wt % PEG-DA, (iii) 8 wt % PEG-DA, and (iv) 10 wt % PEG-DA.

FIGS. 8A-8D show results from evaluating thermally or mechanically induced protein unfolding in an exemplary 3D printed bioplastic object, wherein FIG. 8A shows the solid state circular dichroism spectra indicating that bovine serum albumin (“BSA”) maintained its conformation after photocuring, but that this structure was lost after thermal cure (120° C.); FIG. 8B shows ATR-FTIR spectra of the amide region, showing the transition of the α-helical structure of BSA into β-sheets after thermal cure; FIG. 8C shows a spectrum obtained from Dynamic Mechanical Thermal Analysis (“DMTA”) for a sample before and after thermal cure, which shows a glass transition at 78° C.; and FIG. 8D shows representative uniaxial tensile stress-strain curves comparing a thermally cured sample (σₘₐₓ=46 MPa) and non-thermally cured sample (σₘₐₓ=24 MPa) wherein the ductility observed in the non-thermally cured sample is lost in the thermally cured sample as a result of the increased presence of intermolecular β-sheets that were formed.

FIG. 9 is a graph of relative heat flow as a function of temperature showing the thermal denaturation of BSA (line A) and BSA modified with PEG-DA (line B).

FIG. 10 is a graph of compressive stress (MPa) as a function of strain (%) showing that the compressive elastic modulus varies from 10.5 MPa for non-heated printed object to 103.5 to heated printed objects and that the compressive strength at the 80% of deformation raises from 75.5 MPa for the dehydrated objects to 334.9 MPa for thermally cured objects.

FIG. 11 is a schematic illustration of a printed object made using a disclosed composition embodiment and further showing the chemical and/or physical state of components in the composition during steps of printing, heating, and deformation.

FIG. 12 is a graph of compressive stress (MPa) as a function of compressive strain (mm/mm) showing that after 10 cycles of compression and recovery of the compressed objects (4 mm×8 mm; thickness×diameter) using heat, the same sample retains the initial compressive modulus with small increases to the compressive modulus with each cycle.

FIG. 13 provides illustrations of different physical and/or mechanical characteristics of a gastrointestinal stent made using a disclosed composition embodiment, the illustrations showing the shape recovery capabilities of the stent under various conditions.

### SEQUENCES

The nucleic and amino acid sequence listed in the accompanying sequence listing is shown using standard letter abbreviations for nucleotide bases, and three letter code for amino acids, as defined in 37 C.F.R. § 1.822. Only one strand of each nucleic acid sequence is shown, but the complementary strand is understood as included by any reference to the displayed strand. The Sequence Listing is submitted as an ASCII text file in the form of the file named “Sequences.txt” (˜3.50 kb), which was created on Dec. 20, 2021, which is incorporated by reference herein. In the accompanying sequence listing:

SEQ ID NO: 1 is the amino acid sequence of a CTPR10 protein.

### DETAILED DESCRIPTION

### I. Overview of Terms

The following explanations of terms are provided to better describe the present disclosure and to guide those of ordinary skill in the art in the practice of the present disclosure. As used herein, “comprising” means “including” and the singular forms “a” or “an” or “the” include plural references unless the context clearly dictates otherwise. The term “or” refers to a single element of stated alternative elements or a combination of two or more elements, unless the context clearly indicates otherwise. Additionally, the term “includes” and “has” have the same meaning as “comprises.” Further, the term “coupled” does not exclude the presences of intermediate elements between the coupled items.

Although the steps of some of the disclosed methods are described in a particular sequential order for convenient presentation, it should be understood this manner of description encompasses rearrangement, unless a particular ordering is required by specific language set forth below. For example, steps described sequentially may in some cases be rearranged or performed concurrently. Additionally, the description sometimes uses terms like “produce” and “provide” to describe the disclosed methods. These terms are high-level abstractions of the actual steps that are performed. The actual steps that correspond to these terms will vary depending on the particular implementation and are readily discernible by one of ordinary skill in the art.

Unless explained otherwise, all technical and scientific terms used herein have the same meaning as commonly understood to one of ordinary skill in the art. The compositions, methods, and examples are illustrative only and not intended to be limiting, unless otherwise indicated. Other features of the disclosure are apparent from the following detailed description and the claims.

The composition and method embodiments described herein should not be construed as limiting in any way. Instead, the present disclosure is directed toward all novel and non-obvious features and aspects of the various disclosed embodiments, alone and in various combinations and sub-combinations with one another. The disclosed apparatus is not limited to any specific aspect or feature or combinations thereof, nor does the disclosed apparatus require that any one or more specific advantages be present, or problems be solved. Any theories of operation are to facilitate explanation, but the disclosed apparatus is not limited to such theories.

Unless otherwise indicated, all numbers expressing quantities of components, molecular weights, percentages, temperatures, times, and so forth, as used in the specification or claims are to be understood as being modified by the term “about.” Accordingly, unless otherwise indicated, implicitly or explicitly, the numerical parameters set forth are approximations that can depend on the desired properties sought and/or limits of detection under standard test conditions/methods. When directly and explicitly distinguishing embodiments from discussed prior art, the embodiment numbers are not approximate unless the word “about” is recited. Ratios, concentrations, amounts, and other numerical data may be expressed herein in a range format. It is to be understood that such a range format is used for convenience and brevity, and thus, should be interpreted in a flexible manner to include not only the numerical values explicitly recited as endpoints of the range, but also to include all the individual numerical values or sub-ranges encompassed within that range. Furthermore, not all alternatives recited herein are equivalents.

For chemical formulas provided herein, the symbol “ [symbol] ” is used to indicate a bond disconnection in any abbreviated structures/formulas provided herein. A person of ordinary skill in the art recognizes that the definitions provided below and the compounds and formulas included herein are not intended to include impermissible substitution patterns (e.g., methyl substituted with 5 different groups, and the like). Such impermissible substitution patterns are easily recognized by a person of ordinary skill in the art. In formulas and compounds disclosed herein, a hydrogen atom is present and completes any formal valency requirements (but may not necessarily be illustrated) wherever a functional group or other atom is not illustrated. Any functional group disclosed herein and/or defined above can be substituted (with a functional group or other groups, as defined below) or unsubstituted, unless otherwise indicated herein.

To facilitate review of the various embodiments of the disclosure, the following explanations of specific terms and abbreviations are provided:

Acrylate Group: A functional group having a formula

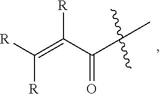

wherein each R independently can be hydrogen, aliphatic, aromatic, or heteroaliphatic; and R^{a} can be hydrogen, aliphatic, aromatic, heteroaliphatic, or any combination thereof. With reference to this formula, the acrylate group can be connected to a heteroaliphatic linking group via the carbonyl carbon atom of the acrylate group (as is intended to be indicated by the wavy line) and a heteroatom of the heteroaliphatic linking group, wherein the heteroaliphatic linking group can be a poly(heteroaliphatic)-based compound as described herein, such as compounds comprising one or more poly(alkylene oxide) groups, poly(alkylene thiol) groups, poly(alkylene amine) groups, and the like.

Aliphatic: A hydrocarbon group having at least one carbon atom to 50 carbon atoms (C₁₋₅₀), such as one to 25 carbon atoms (C₁₋₂₅), or one to ten carbon atoms (C₁₋₁₀), and which includes alkanes (or alkyl), alkenes (or alkenyl), alkynes (or alkynyl), including cyclic versions thereof, and further including straight- and branched-chain arrangements, and all stereo and position isomers as well.

Aromatic: A cyclic, conjugated group or moiety of, unless specified otherwise, from 5 to 15 ring atoms having a single ring (e.g., phenyl) or multiple condensed rings in which at least one ring is aromatic (e.g., naphthyl, indolyl, or pyrazolopyridinyl); that is, at least one ring, and optionally multiple condensed rings, have a continuous, delocalized π-electron system. Typically, the number of out of plane π-electrons corresponds to the Hückel rule (4n+2). The point of attachment to the parent structure typically is through an aromatic portion of the condensed ring system. For example,

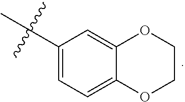 [SMILES: *c1ccc2c(c1)OCCO2]

However, in certain examples, context or express disclosure may indicate that the point of attachment is through a non-aromatic portion of the condensed ring system. For example,

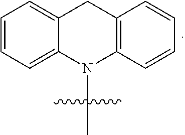 [SMILES: *N1c2ccccc2Cc2ccccc21]

An aromatic group or moiety may comprise only carbon atoms in the ring, such as in an aryl group or moiety, or it may comprise one or more ring carbon atoms and one or more ring heteroatoms comprising a lone pair of electrons (e.g. S, O, N, P, or Si), such as in a heteroaryl group or moiety.

Coupled: The term “coupled” means joined together, either directly or indirectly. A first atom or molecule can be directly coupled or indirectly coupled to a second atom or molecule.

Diacrylate-Containing Compound: A compound comprising at least two acrylate groups. In particular embodiments, the diacrylate-containing compound comprises a polymeric linking group bound to at least two diacrylate groups, such as a poly(heteroaliphatic) linking group. In some embodiments, a diacrylate-containing compound can comprise one or more additional acrylate groups beyond the requisite two acrylate groups. For example, some embodiments of the present disclosure can involve a diacrylate-containing compound comprising a third acrylate group (e.g., a triacrylate-containing compound) or a fourth acrylate group (e.g., a tetraacrylate-containing compound). In such embodiments, additional acrylate groups can be bound to a carbon atom of the diacrylate-containing compound.

Energy Source: A source that delivers photons, ions, electrons, heat, or other suitable energy sufficient for inducing polymerization. In some embodiments, the energy source may be configured to project and/or have differing wavelengths, pathways, and/or images, both two- and three-dimensional.

Free radical: An atom, molecule, or ion with an unpaired electron. Free radicals are formed by splitting a chemical bond within a molecule, and are usually short-lived and highly reactive. Free radicals are capable of initiating chemical chain reactions, e.g., polymerization. They also act as initiators or intermediates in oxidation, combustion, and photolysis.

Functional group: A specific group of atoms within a molecule that can be responsible for chemical reactions of the molecule. Exemplary functional groups include, without limitation, halo (fluoro, chloro, bromo, iodo), epoxide, hydroxyl, carbonyl, aldehyde, carbonate ester, carboxylate, carboxyl, ether, ester, peroxy, hydroperoxy, carboxamide, amino (primary, secondary, tertiary), ammonium, imide, azide, cyanate, isocyanate, thiocyanate, nitrate, nitrite, nitrile, nitroalkyl, nitroso, pyridyl, phosphate, sulfonyl, sulfide, thiol (sulfhydryl), disulfide.

Haloaliphatic: An aliphatic group wherein one or more hydrogen atoms, such as one to 10 hydrogen atoms, independently is replaced with a halogen atom, such as fluoro, bromo, chloro, or iodo.

Haloheteroaliphatic: A heteroaliphatic group wherein one or more hydrogen atoms, such as one to 10 hydrogen atoms, independently is replaced with a halogen atom, such as fluoro, bromo, chloro, or iodo.

Heteroaliphatic: An aliphatic group comprising at least one heteroatom to 20 heteroatoms, such as one to 15 heteroatoms, or one to 5 heteroatoms, which can be selected from, but not limited to oxygen, nitrogen, sulfur, silicon, boron, selenium, phosphorous, and oxidized forms thereof within the group. Alkoxy, alkylene oxide, alkylene thiol, alkylene amine, ether, amino, disulfide, peroxy, and thioether groups are exemplary (but non-limiting) examples of heteroaliphatic.

Hydrogel: A cross-linked three-dimensional network of polymeric chains that are capable of absorbing and retaining molecules (e.g., water, polar solvents, non-polar solvents, drugs in liquid form, or the like) in their three-dimensional networks. Hydrogel-forming polymeric chains can comprise one or more hydrophilic functional groups in their polymeric structures, such as amino (NH₂), hydroxyl (OH), amide (—CONH—, —CONH₂), sulfate (—SO₃H), or any combination thereof. In some embodiments, the polymeric chains can comprise a plurality of the same monomeric units. In other embodiments, the polymeric chains can comprise a plurality of different monomeric units.

Non-Acrylated Protein: A native or synthesized/engineered protein that comprises one or more primary amine group (e.g., as provided by, for example, a lysine residue), wherein none the primary amine groups is functionalized with an acrylic acid group or methacrylic acid group (or anionic form thereof) prior to exposure to any other reagents included in a composition comprising the protein, and particularly any diacrylate-containing compound included in the composition. In the present disclosure, the protein used in the disclosed composition is in the form of a non-acrylated protein prior to exposure to any other reagents included in the composition, and particularly any diacrylate-containing compound.

Photoinitiating Component: A compound, or combination of compounds, that is capable of initiating or promoting the formation of one or more radical species and/or ionic species from a diacrylate-containing compound and/or a saturated or partially saturated form thereof. In some embodiments, the photoinitiating component can be a single compound, or a combination of compounds, that facilitate polymerization.

Poly(alkylene amine)-Diacrylate Compound: A compound comprising one or more poly(alkylene amine) groups (e.g., —[N—R^{a}]ₙ, wherein R^{a} is an aliphatic group, such as an ethylene group; and n is an integer of at least 2) that are bound to at least two acrylate groups.

Polymerization: A chemical reaction, usually carried out with a catalyst, heat, light, or a combination thereof, in which a large number of relatively simple molecules (e.g., monomers) combine to form a chainlike macromolecule (a polymer). The chains further can be combined, or crosslinked, by the addition of appropriate chemicals. The monomers typically are unsaturated or otherwise reactive substances. In some embodiments, polymerization involves addition or condensation. In particular embodiments, polymerization occurs when an initiator, usually a free radical, reacts with a double bond in the monomer. The free radical adds to one side of the double bond, producing a free electron on the other side. This free electron then reacts with another monomer. In such embodiments, the monomers can be the diacrylate-containing compound and the protein.

Poly(alkylene thiol)-Diacrylate Compound: A compound comprising one or more poly(alkylene thiol) groups (e.g., —[S—R^{a}]ₙ, wherein R^{a} is an aliphatic group, such as an ethylene group; and n is an integer of at least 2) that are bound to at least two acrylate groups.

Protein: A biomolecule comprising one or more chains of amino acid residues. In particular embodiments, the protein embodiments of the present disclosure are globular proteins, which comprise a tertiary structure that provides a round, or spherical, or spherical-like structure (or a non-fibrous structure). In an independent embodiment, the protein embodiments of the present disclosure are not fibrous proteins.

Saturated Form of a Diacrylate-Containing Compound: This phrase refers to a diacrylate-containing compound that has been covalently coupled to a protein functional group through a carbon atom of one of the two double bonds of the diacrylate-containing compound such that at least one of the double bonds of the diacrylate-containing compound is no longer present because it is coupled to the protein.

Thermoplastic: Refers to a material that softens when heated and hardens when cooled.

Thermoset: Refers to a material that is comprised of polymeric networks that are bound by covalent bonds.

Viscosity Index (VI): A measure of the change in viscosity with variations in temperature. A greater viscosity index indicates a smaller viscosity change with temperature.

### II. Introduction

Bio-sourced materials that can supplant petroleum-based materials are valuable components of sustainability. Moreover, renewable materials with greater complexity and functionality can meet the demands for the full spectrum of applications from aerospace to medicine. Certain protein-based materials, such as biologically derived proteins, engineered proteins, and polymer-protein conjugates, have been developed to create materials that emulate biological and nonbiological function, as well as other physical characteristics (e.g., mechanical properties). However, their application is restricted by poor processability into 3D form as well as factors and limitations in mechanical performance.

Both 3D and 4D additive manufacturing (AM) processes that utilize vat photopolymerization have tremendous potential in industrial manufacturing for the future production of parts and supplies. While a growing list of elastomers, plastics, and composites have been reported, there are relatively few examples of compositions that are bio-sourced and bio-degradable. Photocurable compositions for vat photopolymerization comprise cross-linkable (e.g., photo-cross-linkable) molecules that have a low viscosity and fast rate of photocuring. In general, a low viscosity (e.g., 0.25 Pa·s to 10 Pa·s) facilitates composition reflow and minimizes the capillary forces exerted onto the printed object during printing. Naturally occurring polymers often require modification with photocurable functionalities to become printable. While 4D printed structures that undergo chemical or physical changes in response to their environment are often inspired by nature, there are relatively few examples that use bio-sourced and biodegradable materials.

Both, structural and globular proteins are known as macromolecules that can introduce material plasticity or elasticity based on the unfolding or disassembly of the proteins. Spider silk is a structural protein that has a specific energy to failure (160 J·g⁻¹) that is greater than Kevlar (50 J·g⁻¹) and is attributed to the presence of β-sheet domains. Single molecule atomic force microscopy (AFM) experiments on titin have shown that proteins can be unraveled reversibly or irreversibly upon the application of a tensile force. Since then, the secondary and tertiary structure of proteins have been utilized to achieve biomaterials with unique combinations of extensibility, strength, and resilience; however, there has not been a demonstration of a protein-based material that exhibits plasticity due to unfolding, with a corresponding shape recovery back to its original form.

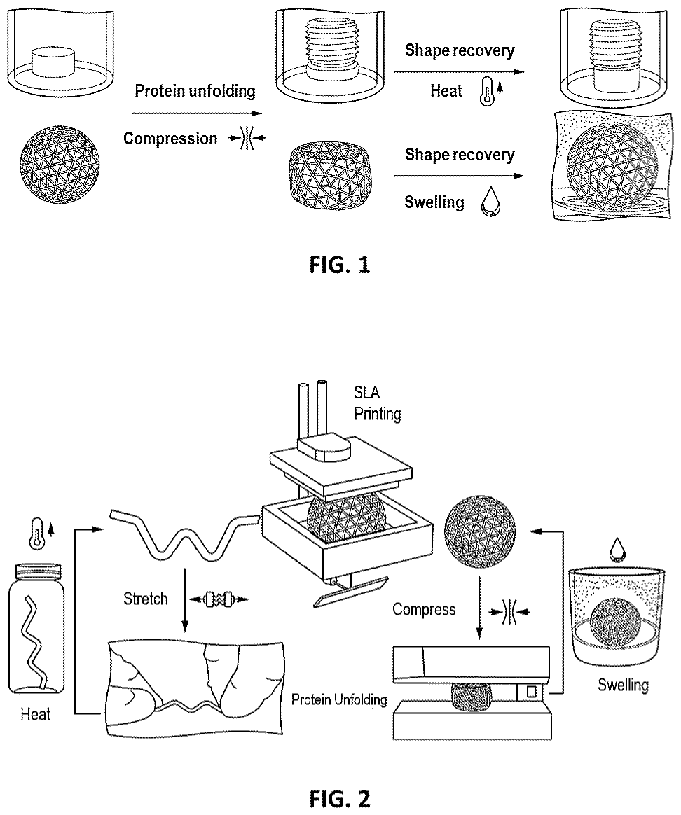

Disclosed herein are embodiments of a protein-based composition for additive manufacturing that affords printed bioplastic objects with shape recovery behavior. For example, see FIGS. 1 and 2, which show representative embodiments where objects were 3D printed and then dried to afford a 3D bioplastic object. In FIG. 1, the printed objects were deformed and then converted back to their original shapes using thermal treatment (top) or swelling in an aqueous solution (bottom). In FIG. 2, the printed objects were plastically deformed by compression or elongation to afford a metastable state, and recovered their original shape upon heating or swelling in an aqueous solution. In an independent embodiment, the proteins do not refold into their globular shape within the printed objects. In other embodiments, proteins do refold into their globular shape within the printed objects. Nevertheless, the rapid shape recovery behavior of these bioplastics, which can occur in a matter of seconds, can be useful for the production of medical devices such as scaffolds, implants, or stents. Features of the present disclosure are described in additional detail herein.

### III. Composition, Object, and Method Embodiments

Disclosed herein are embodiments of a composition for use in additive manufacturing techniques, such as processes utilizing vat photopolymerization, which do not require a separate step to functionalize the main starting material. In particular embodiments, the main starting material used in the composition is a protein, such as a globular protein, which is reacted with a diacrylate-containing compound to form the printed object embodiments disclosed herein. In embodiments disclosed herein, the protein is not functionalized with acrylic acid or methacrylic acid prior to reacting it with the diacrylate-containing compound. As such, a non-acrylated protein is used in particular embodiments. In particular embodiments, the protein used in the composition is not, or is other than, BSA-methacrylate (or “BSA-MA”) or BSA-acrylate. Method embodiments also are disclosed. Materials (e.g., 3D printed bioplastics and/or hydrogels) made using the composition embodiments are also disclosed herein. The printed objects can exhibit mechanical properties that utilize the stored length of the protein macromolecule and thus create a closed loop life cycle with bioplastics.

In particular embodiments, the composition is a globular protein-based composition for additive manufacturing using photopolymerization. Proteins that can be used include, but are not limited to, globular proteins, such as an albumin protein (e.g., a serum albumin protein, a lactalbumin protein, a whey protein, an ovalbumin protein, or the like), a pepsin protein, a hemoglobin protein, an enzyme, a lysozyme, or a combination thereof. In some embodiments, the globular protein is a CTPR10 protein having a sequence according to SEQ ID NO: 1. In particular embodiments, the protein is water-soluble. The native conformation of the globular proteins is largely retained in the 3D printed objects, such as the 3D bioplastic and/or hydrogel materials discussed herein. Within a photopolymerized network of globular proteins, each protein molecule can be unfolded to release a “stored length” as a mechanism of energy dissipation. For example, the protein molecules possess a “stored length” that can be revealed during mechanical deformation (e.g., extension/stretching or compression) of a 3D bioplastic object that comprises the protein molecules. While such plastically deformed objects can retain this state for an indefinite period of time, heating the object or submersing in water can facilitate returning the deformed object to its original 3D shape, such as additively manufactured printed shapes as illustrated in FIG. 1. In some embodiments, incorporation of plasticity within a cross-linked thermoset affords a mechanism for shape recovery after the object has been plastically deformed.

Composition embodiments disclosed herein further comprise a diacrylate-containing compound. In some embodiments, the diacrylate-containing compound is a water-soluble compound that is used a co-reactant in aqueous formulations comprising the protein. In some embodiments, the diacrylate-containing compound can comprise a polymeric heteroaliphatic linking group bound to two or more acrylate groups. The diacrylate-containing compound can have a formula R₂C═CR—C(═O)—[X—R^{a}]ₙ—X—C(═O)—C(R)═CR₂, wherein each R independently can be hydrogen, aliphatic, aromatic, haloaliphatic, haloheteroaliphatic, or heteroaliphatic; each R^{a} independently can be aliphatic, aromatic, haloaliphatic, haloheteroaliphatic, heteroaliphatic, or any combination thereof; each X independently can be oxygen, sulfur, or NR^{b} (wherein R^{b} is hydrogen, aliphatic, haloaliphatic, haloheteroaliphatic, heteroaliphatic, or aromatic); and n is an integer of at least 2 or more. In particular embodiments, each R independently is hydrogen or aliphatic; each R^{a} is an ethylene group; each X is oxygen, sulfur, or NH; and n is an integer selected from 2 or more. In some embodiments, the diacrylate-containing compound has a formula of R₂C═CR—C(═O)—O—R^{a}—O—C(═O)—C(R)═CR₂, wherein each R independently can be hydrogen, aliphatic, aromatic, haloaliphatic, haloheteroaliphatic, heteroaliphatic, or any combination thereof; R^{a} can be aliphatic, aromatic, haloaliphatic, haloheteroaliphatic, heteroaliphatic, or any combination thereof; and further acrylate groups can be bound to R^{a} via an aliphatic or heteroaliphatic linker group. Non-limiting examples of water-soluble diacrylate-containing compounds can include poly(heteroaliphatic)-based compounds functionalized with two or more acrylate groups, such as poly(alkylene oxide) diacrylate compounds, poly(alkylene thiol) diacrylate compounds, poly(alkylene amine) diacrylate compounds, and the like. In particular embodiments, the water-soluble diacrylate-containing compound is a poly(ethylene glycol) diacrylate compound (“PEG-DA”) having molecular weights (Mₙ) that range from 300 g/mol to 10,000 g/mol, such as Mₙ 700 g/mol. In some embodiments, the diacrylate-containing compound can be a multi-functional acrylate compound that comprises further acrylate moieties in addition to the two acrylates of the diacrylate compound. Examples of multi-functional acrylates (e.g., diacrylate-containing compounds that comprise one or two additional acrylate groups) that can be included in the aqueous composition include, but are not limited to, compounds comprising three or four acrylate-functionalized poly(alkylene oxide) branches (also known in the art as 3-arm or 4-arm PEG acrylate groups), or three or four acrylate-functionalized poly(alkylene thiol) groups, or three or four acrylate-functionalized poly(alkylene amine) groups. Exemplary multi-functional acrylates can include, but are not limited to, PEG-3 trimethylolpropane triacrylate, ethoxylated (4) pentaerythritol tetra-acrylate, and combinations thereof.

In additional embodiments, the composition further comprises a photoinitiating component. The photoinitiating component can be any molecule (or combination of molecules) known to those of ordinary skill in the art with the benefit of the present disclosure to produce a radical species when exposed to a certain wavelength of light, such as a wavelength ranging from 350 nm to 420 nm, such as 360 nm to 410 nm, or 365 nm to 405 nm. Photoinitiating components that can generate radicals during the photocuring process can include, but are not limited to, ruthenium trisbipyridine chloride, lithium phenyl-2,4,6-trimethylbenzoylphosphinate (LAP), 2-hydroxy-2-methylpropiophenone, benzophenone, ethyl 2,4,6-trimethylbenzoylphenyl phosphinate, 1-hydroxy-cyclohexyl-phenyl-ketone, 2-hydroxy-2-methyl-1-phenyl-1-propanone, 2,2-dimethoxy-2-phenyl acetophenone, 2-hydroxy-1-[4-(2-hydroxyethoxy)phenyl]-2-methyl-1-propanone, and diphenyl (2,4,6-trimethylbenzoyl)-phosphine oxide, with any of these being used alone or in combination with sodium persulfate (“SPS”), ammonium persulfate, or a combination thereof. In particular embodiments, the photoinitiating components can include, but are not limited to ruthenium trisbipyridine chloride, lithium phenyl-2,4,6-trimethylbenzoylphosphinate (LAP), or 2-hydroxy-2-methylpropiophenone, with any of these being used alone or in combination with SPS, ammonium persulfate, or a combination thereof. Representative embodiments include ruthenium trisbipyridine chloride in combination with SPS.

The amount of the globular protein used in the composition embodiments disclosed herein can be selected so as to retain a relatively low viscosity at high concentrations. In some embodiments, the globular protein is used at an amount sufficient to retain a viscosity of the composition that is 10 Pa·s or less, such as greater than 0 Pa·s to less than 10 Pa·s, or greater than 0 Pa·s to less than 8 Pa·s, or greater than 0 Pa·s to less than 6 Pa·s, or greater than 0 Pa·s to less than 4 Pa·s, or greater than 0 Pa·s to less than 2 Pa·s, or greater than 0 Pa·s to less than 1 Pa·s, particularly at protein concentrations greater than 15% by weight (e.g., greater than 15% to 40% by weight, such as 20% to 35% by weight, or 25% to 35% by weight). In particular embodiments, the viscosity is 10 Pa·s or less. In some embodiments, maintaining a low viscosity of the printable composition can facilitate the ability of the composition to settle during printing (e.g., between layers), thereby increasing the speed at which printing can take place and the level of fine detail that can be captured. In some embodiments, the non-acrylated globular protein and the diacrylate-containing compound are present in the composition at a ratio ranging from 1:9 to 9:1 (non-acrylated globular protein to diacrylate-containing compound), such as at a ratio ranging from 1:3 to 3:1. In some embodiments, the photoinitiating component is present in an amount between 0 wt % and 20 wt % of the total weight of the composition, such as greater than 0 wt % to 15 wt %, or 0.5 wt % to 10 wt %, or 1 wt % to 5 wt %, or 1 wt % to 3 wt %. The composition can be made by dissolving the diacrylate-containing compound in water, followed by adding the globular protein with mixing to provide an initial reaction mixture. Then, a solution of the photoinitiating component can be added to the initial reaction mixture to provide a photopolymerizable mixture. In embodiments where two components make up the photoinitiating component (e.g., a persulfate compound and a catalyst), an aqueous solution of the catalyst can be added to the initial reaction mixture, followed by an aqueous solution of the persulfate compound.

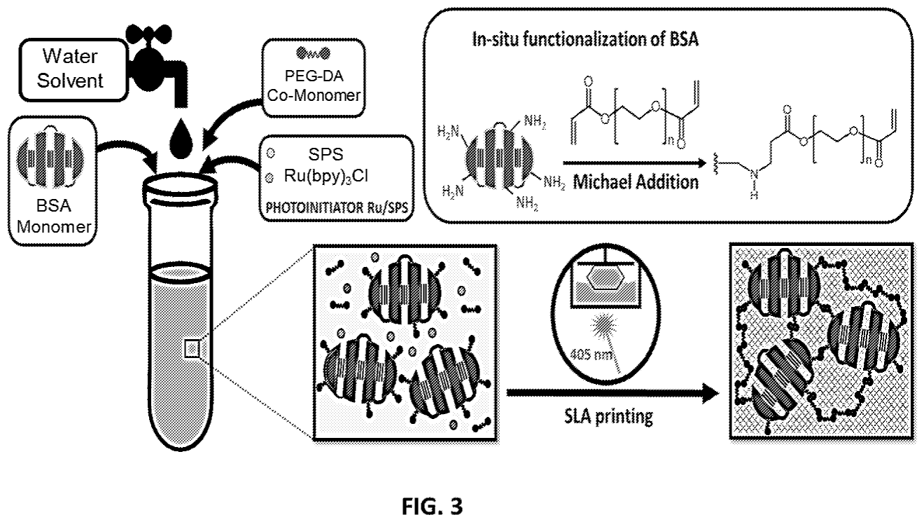

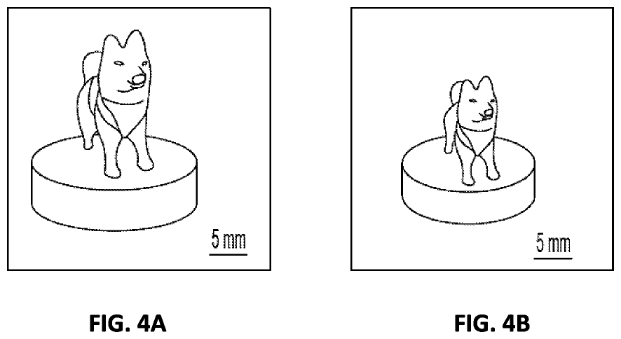

Using the diacrylate-containing compound as a reactive monomer in combination with the globular protein (e.g., a non-acrylated protein) provides a composition that can undergo a rapid photo-initiated transformation into 3D printed object during the printing process, which can be patterned in some embodiments. In particular embodiments, the globular protein is modified in situ with the diacrylate-containing compound via an aza-Michael addition, which can be facilitated or initiated using the photoinitiating component so as to provide a protein modified with the acrylate groups of the diacrylate-containing compound. The protein and the diacrylate-containing compound are joined together through a covalent bond formed between an amine group of the globular protein (e.g., an amine group of one or more lysine moieties of the protein) and a carbon atom of the diacrylate-containing compound, which typically is a carbon atom of one of the olefins of the acrylate group. Due to the reaction between the protein and the acrylate group, the acrylate group is converted to a saturated form of the acrylate moiety. This reactivity is illustrated, for example, in FIG. 3. Such binding can occur between two separate protein molecules and one diacrylate-containing compound, a single protein molecule and one diacrylate-containing compound, a single protein molecule and two or more diacrylate-containing compounds, and any other feasible combinations. The binding between the protein and at least one diacrylate-containing compound facilitates forming a polymerized network within printed objects obtained by printing composition embodiments disclosed herein. In particular embodiments, the polymerized network can comprise a first globular protein molecule that is covalently bound to a saturated form of a first diacrylate-containing compound, wherein the protein is directly covalently bound to a carbon atom of the saturated form of the first diacrylate-containing compound via a native functional group of the protein (e.g., a non-acrylated amine group); and wherein the saturated form of the diacrylate-containing compound is further directly covalently bound to a hydrogen atom, a saturated form of a second diacrylate-containing compound, a second globular protein molecule, or a combination thereof. In some embodiments, the as-printed 3D object obtained by printing composition embodiments disclosed herein is a hydrogel. In particular embodiments, the hydrogel can de-swell isotropically upon dehydration to afford a 3D bioplastic object. As such, in some embodiments, the printed object can be a hydrogel, a bioplastic material, or a combination thereof. In some embodiments, the bioplastic material retains the same or substantially similar geometries and shapes as the printed hydrogel, but with 30% smaller dimensions. For example, see FIGS. 4A and 4B, wherein FIG. 4A shows the hydrogel form and FIG. 4B shows the bioplastic form. In some embodiments, the bioplastic material can comprise 75% protein.

Also disclosed herein are embodiments of a method of making objects from the composition. In particular embodiments, the method comprises printing an object using a composition comprising (i) a non-acrylated globular protein; (ii) a diacrylate-containing compound; (iii) a photoinitiating component; and (iv) a solvent, wherein the printing is carried out using an additive manufacturing device that uses an energy source to promote vat photopolymerization. Any suitable additive manufacturing process that uses liquid resins in combination with an energy source, typically a light source, can be used to print the object using the composition. In some embodiments, the additive manufacturing process is a printing process that uses vat polymerization, such as laser-scanning stereolithography (“SLA”) printing, digital light projection (“DLP”), daylight polymer printing, continuous liquid interface production (“CLIP”), high-area rapid printing (“HARP”) and the like. Steps used in such printing techniques are known to individuals having ordinary skill in the art with the benefit of the present disclosure. The composition embodiments disclosed herein can be used as the photopolymerizable resin used in such printing techniques. In some embodiments, the method further comprises drying the object. In additional embodiments, the method can further comprise exposing the object to heat; (ii) hydrating the object; (iii) deforming the object; or (iv) a combination of (i), (ii), and/or (iii). In particular embodiments, the method comprises exposing the object to heat after the object is printed and after any drying step, and then deforming the object after it has been exposed to heat. In some embodiments, exposing the object to heat can enhance mechanical properties of the object, such as the tensile strength of the material. In particular embodiments, the object is exposed to temperatures ranging from higher than ambient temperature to a temperature below any degradation temperature of the globular protein. In some embodiments, the temperature can range from 80° C. to 200° C., such as 100° C. to 200° C., or 125° C. to 200° C., or 150° C. to 200° C., or 175° C. to 200° C. In some embodiments, deforming the object can comprise exposing the object to a compressive force or a tensile force. Deforming the object can facilitate improving mechanical properties of the object, such as the tensile strength of the material, and/or can promote shape recovery behavior of the object. In some embodiments, the object can be exposed to both heat and a deforming force to enhance mechanical properties of the object, such as tensile strength, and/or shape recovery.

In particular exemplary embodiments, the composition is a bovine serum albumin (BSA)-based composition for stereolithographic apparatus (SLA) 3D printing. BSA is a globular protein comprised of a single macromolecular chain that is precisely folded to comprise 67% alpha helices and 17 disulfide bonds. It also exhibits excellent aqueous solubility and low viscosity at various concentrations, such as up to 30 wt %. Given the large molecular weight of BSA (66 kDa), there is great potential for unfolding individual protein chains, as exhibited by embodiments disclosed herein. In particular embodiments, an aqueous composition comprising non-acrylated BSA and PEG-DA was used. The composition further comprises a combination of SPS and Ru(bpy)₃Cl as the photoinitiating component.

In particular embodiments, the BSA composition described above is printed with a Form 2 printer. A rheometer can be used to characterize the viscosity and rate of photo-curing based on storage modulus to provide the printed object. In particular embodiments, the rate of curing used in disclosed method embodiments is insufficient to afford mechanically stable networks during certain embodiments of the printing process resulting solely from photo-induced crosslinking of native residues, such as tyrosine moieties, present in the globular protein. As such, in some embodiments of the present disclosure, any tyrosine moieties of the globular proteins used in the disclosed composition embodiments are not crosslinked with any other tyrosine moieties of the globular protein in any printed object and/or any tyrosine moieties of another globular protein that may be present in the composition or any printed object. If any such crosslinking exists in a printed object using a composition embodiment disclosed herein, then there further exists at least one covalent bond between the globular protein and a saturated form of a diacrylate-containing compound. In exemplary embodiments, the printed object comprises a first globular protein molecule that is a BSA molecule that is covalently bound to a saturated form of a first PEG-diacrylate compound such that the BSA molecule is directly covalently bound to a carbon atom of the saturated form of the first PEG-diacrylate compound via an amine group of the BSA molecule; and wherein the saturated form of the first PEG-diacrylate compound is further directly covalently bound to a hydrogen atom, a saturated form of a second PEG-diacrylate compound, a second BSA molecule, or a combination thereof. As represented in FIG. 3 (which shows a representative schematic showing composition preparation, wherein the composition comprises BSA and PEG-DA), it is currently believed (without being bound to a single theory) that lysine residues on the surface of the BSA react with the acrylates of PEG-DA via an aza-Michael addition. FIG. 3 further shows representative embodiments for formulation of a photopolymerizable composition comprising BSA, PEG-DA, SPS, Ru(bpy)₃Cl, and water; and the in situ aza-Michael addition reaction believed to occur between the surface lysine amines of BSA and the acrylates of PEG-DA (excess).

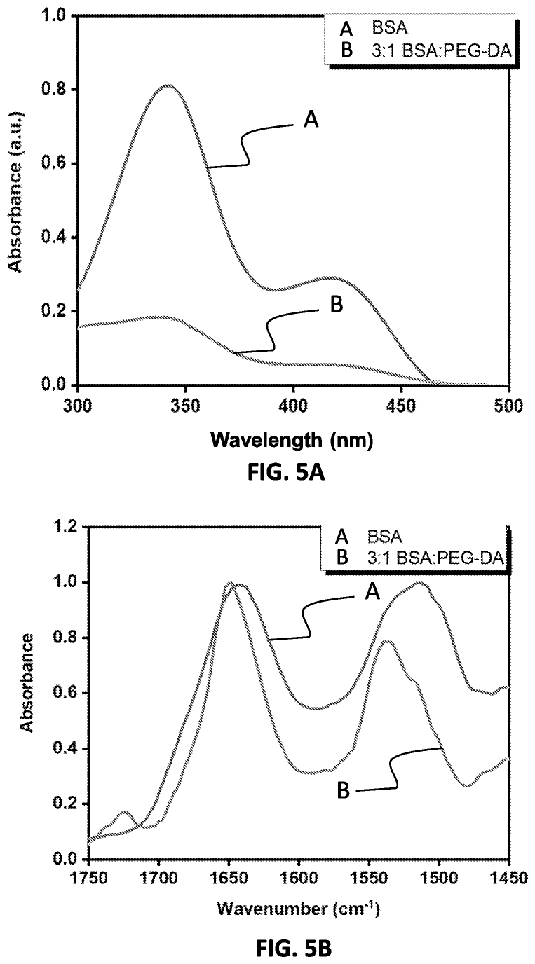

The binding between the non-acrylated globular protein and the diacrylate-containing compound for particular embodiments can be evaluated by using characterization methods sufficient to detect the presence of secondary amines (e.g., IR spectroscopy). In some embodiments, the binding is confirmed by the absence of reactive amines in a 2,4,6-trinitrobenzenesulfonate (TNBS) assay, and the appearance of a peak near 1530 cm⁻¹ corresponding to the secondary amines (N—H in-plane bending) at the IR spectrum (e.g., see FIGS. 5A and 5B). Thus, in particular embodiments, the globular protein (e.g., BSA) is functionalized in situ with acrylates. In some embodiments, functionalizing the globular protein in situ facilitates a significantly faster photocuring rate while keeping the low viscosity that facilitates the composition flow around the build platform during printing and forming new layers.

### IV. Overview of Several Embodiments

Disclosed comprising a non-acrylated globular protein; a diacrylate-containing compound; a photoinitiating component; and a solvent.

In any or all of the above embodiments, the composition of claim 1, wherein the composition has a viscosity that ranges from greater than 0 Pa·s to less than 10 Pa·s.

In any or all of the above embodiments, the non-acrylated globular protein and the diacrylate-containing compound are present at a ratio of 1:9 to 9:1 (non-acrylated globular protein to diacrylate-containing compound).

In any or all of the above embodiments, the non-acrylated globular protein and the diacrylate-containing compound are present at a ratio of 1:3 to 3:1 (non-acrylated globular protein to diacrylate-containing compound).

In any or all of the above embodiments, the non-acrylated globular protein is an albumin protein, a pepsin protein, a hemoglobin protein, an enzyme, a lysozyme, or a combination thereof.

In any or all of the above embodiments, the diacrylate-containing compound is a diacrylate-containing poly(heteroaliphatic) polymer.

In any or all of the above embodiments, the diacrylate-containing compound is a poly(alkylene oxide)-diacrylate compound, a poly(alkylene amine)-diacrylate compound, a poly(alkylene thiol)-diacrylate compound, or a combination thereof.

In any or all of the above embodiments, the photoinitiating component comprises ruthenium trisbipyridine chloride, lithium phenyl-2,4,6-trimethylbenzoylphosphinate (LAP), 2-hydroxy-2-methylpropiophenone, benzophenone, ethyl 2,4,6-trimethylbenzoylphenyl phosphinate, 1-hydroxy-cyclohexyl-phenyl-ketone, 2-hydroxy-2-methyl-1-phenyl-1-propanone, 2,2-dimethoxy-2-phenyl acetophenone, 2-hydroxy-1-[4-(2-hydroxyethoxy)phenyl]-2-methyl-1-propanone, and diphenyl (2,4,6-trimethylbenzoyl)-phosphine oxide, sodium persulfate, ammonium persulfate, or any combination thereof.

In any or all of the above embodiments, the solvent is water.

In any or all of the above embodiments, the photoinitiating component is present in an amount between 0 wt % and 20 wt % of the diacrylate-containing compound.

In any or all of the above embodiments, the non-acrylated globular protein is covalently bound to the diacrylate-containing compound through a covalent bond formed between an amine group of the non-acrylated globular protein and a carbon atom of the diacrylate-containing compound.

In any or all of the above embodiments, the non-acrylated globular protein is non-acrylated bovine serum albumin; the diacrylate-containing compound is a diacrylate-containing poly(ethylene glycol) compound; the photoinitiating component comprises sodium persulfate and ruthenium tris(bipyridyl) chloride; and the solvent is water.

Also disclosed herein is a printed object, comprising a polymerized network comprising a first globular protein molecule that is covalently bound to a saturated form of a first diacrylate-containing compound, wherein the protein is directly covalently bound to a carbon atom of the saturated form of the first diacrylate-containing compound via a non-acrylated functional group of the protein; and wherein the saturated form of the diacrylate-containing compound is further directly covalently bound to a saturated form of a second diacrylate-containing compound, a second globular protein molecule, or a combination thereof.

In any or all of the above embodiments, the first globular protein molecule is a bovine serum albumin molecule that is covalently bound to a saturated form of a first PEG-diacrylate compound such that the bovine serum albumin molecule is directly covalently bound to a carbon atom of the saturated form of the first PEG-diacrylate compound via an amine group of the bovine serum albumin molecule; and wherein the saturated form of the first PEG-diacrylate compound is further directly covalently bound to a saturated form of a second PEG-diacrylate compound, a second bovine serum albumin molecule, or a combination thereof.

In any or all of the above embodiments, the printed object exhibits shape recovery behavior.

Also disclosed are embodiments of a method, comprising printing an object, such as the printed object according to any or all of the above printed object embodiments, using a composition comprising (i) a non-acrylated globular protein; (ii) a diacrylate-containing compound; (iii) a photoinitiating component; and (iv) a solvent, wherein the printing is carried out using an additive manufacturing device that uses an energy source to promote vat photopolymerization or a composition according to any or all of the above composition embodiments.

In any or all of the above embodiments, the method further comprises drying the object.

In any or all of the above embodiments, the additive manufacturing device is a printer capable of stereolithography, digital light processing, continuous liquid interface production, high-area rapid printing, daylight polymer printing, or a combination thereof.

In any or all of the above embodiments, the method further comprises (i) exposing the object to heat; (ii) hydrating the object; (iii) deforming the object; or (iv) a combination of (i), (ii), and/or (iii).

In any or all of the above embodiments, the method comprises exposing the object to heat after the object is printed and after any drying step, and then deforming the object after it has been exposed to the heat.

### V. Examples

General Methods—All materials were purchased from Sigma-Aldrich unless otherwise stated. Tris(2,2′-bipyridyl)-dichlororuthenium(II) hexahydrate (Ru(bpy)₃Cl) (99.95%), poly(ethylene glycol) diacrylate (Mn=700 g/mol), and sodium persulfate (SPS) were used as received. Ultrapure and ultralow fatty acid content BSA was purchased from Nova Biologics. Formlabs standard clear resin (FLGPCL04) was purchased from Formlabs. Ultraviolet-visible spectra were recorded with a Varian Cary 5000 UV-Vis-NIR spectrophotometer and a 1 cm path quartz cuvette, and data collection and analysis were carried out with Cary WinUV software. Thermal analyses were conducted on a Mettler Toledo DSC 3+ differential scanning calorimeter (DSC). All scans were carried out in hermetic aluminum pans under a nitrogen atmosphere for sample weights between 5 and 10 mg. For the purpose of studying transition temperatures, scans were performed from 50° C. to 200° C. with a scan rate of 10° C./minute. Attenuated Total Reflection Fourier Transform Infrared (ATR-FTIR) spectroscopy was performed with a Perkin Elmer Frontier FTIR spectrometer with a mounted Universal ATR Sampling Accessory. Spectra were taken with a resolution of 4 cm⁻¹ and were averaged over 32 scans in the 4000-400 cm⁻¹ range, and data collection and analysis were carried out with PerkinElmer Spectrum software.

Protein-Based Composition Preparation Protocol—The weight percentages are based on the total components of the composition, including the aqueous solvent. As a representative example, a preparation of 20 g of composition with 30 wt % BSA and 10 wt % PEG-DA is described. First, 2 g of PEG-DA were dissolved in 11.2 mL of DI water, then 6 g of BSA was slowly added to this solution with gentle mixing until well dissolved. Next, 15 mg Ru(bpy)₃Cl dissolved in 400 μL of DI water and 48 mg SPS dissolved in 400 μL of DI water were sequentially dissolved into the composition. The final composition was covered in aluminum foil and stored at 4° C. until use. To prepare other formulations, the same procedure was followed, changing only the comonomer and DI water quantities.

Rheology—Rheology measurements were performed on a TA Instruments Discovery Hybrid Rheometer-2. Viscosity versus shear rate experiments were performed at a shear rate increasing from 1 to 100 s⁻¹ using a 40 mm cone and plate geometry with a cone angle of 1.019°, a solvent trap, and a gap height of 26 μm. To conduct photorheology experiments, a 365 nm LED UV-curing accessory with disposable acrylic plates was used. The photorheological experiments were conducted using constant 1% strain and a frequency of 1 Hz, and a 20 mm parallel plate with a gap height of 1000 μm. A 60 s dwell time elapsed before the UV lamp was turned on for 5 min. Data collection and analysis were carried out with TRIOS software (TA Instruments).

3D Printing—A Formlabs Form 2 printer was used to fabricate the 3D printed constructs, but the build plate and composition tray were modified to reduce the total volume of composition required to print. The build plate was cut down to 45 mm×45 mm, and a 48 mm×78 mm×28 mm border was 3D printed on a Flashforge Creator Pro, then glued to the composition tray to form a small reservoir within the standard composition tray. 3D constructs were designed with Autodesk Fusion 360 or downloaded from Thingiverse. The composition was poured into the reservoir, and the bioplastic constructs were then printed in the Form 2's Open Mode with a layer height of 100 μm. The Form 2 SLA printer uses a 405 nm violet laser (250 mW) with a laser spot size of 140 microns. The duration of the light exposure for each layer was approximately 30 seconds. Upon completion of the print, samples were removed from the build plate, rinsed in DI water to remove any uncured composition, and then air-dried. No post-curing process was needed. Some samples were further thermally cured by placing them in a 120° C. oven for 180 minutes.

Circular Dichroism—The secondary structure of proteins within the composition was determined by circular dichroism using a Jasco J-815 spectropolarimeter. The protein-based composition was prepared at a final protein concentration of 100 μM, while maintaining the relative concentrations of the other components (e.g., 10 wt % PEG-DA+30 wt % BSA+0.075 wt % Ru(bpy)₃Cl+0.24 wt % SPS+59.685 wt % DI water). A thin film of the protein-based composition was then formed on a 1 cm² quartz slide, by spin-coating 40 μL of the material at an angular speed of 3000 RPM for 10 min on a Laurell Technologies corporation Model WS-400B-6NPP/LITE spin-coater. The newly formed thin film was immediately UV-cured for 1.5 hours, using a 405 nm lamp at maximum power placed at a distance of 2 cm. A thermal treatment was then performed on the solid film, heating the quartz slide at 120° C. for 1 hour. The CD spectrum of the material was monitored after each step, using 1 nm increments and 8 second integration time over a wavelength range of 190 to 260 nm.

Mechanical Properties—Tensile tests were performed with a TA Electroforce TestBench uniaxial tension instrument at a speed of 6 mm/minutes until mechanical failure of the sample. The dogbones were 3D printed in the Form2 SLA printer, following ISO 527-2 (ISO 527-2/5B/6). Cylindrical compression samples (10 mm diameter×5 mm height) were 3D printed and dried, before being tested with an Instron 5585H 250 kN electro-mechanical test frame with a 50 kN load cell using a crosshead rate of 1.3 mm/minute. All the specimens were first air-dried overnight, and then vacuum-dried during at least 24 hours prior to the mechanical test.

Thermal Transitions—The steady state mechanical behavior of the samples was determined by dynamic mechanical thermal analysis (DMTA) measurements, conducted in compression mode in a Dynamic Mechanical Analyzer, Triton 2000 DMA (Triton Technology). Cylindrical samples (4.1 mm×7.6 mm; thickness×diameter) were heated from −50° C. to 150° C. at a constant heating rate of 4° C./minute and at a frequency of 1 Hz.

### Example 1

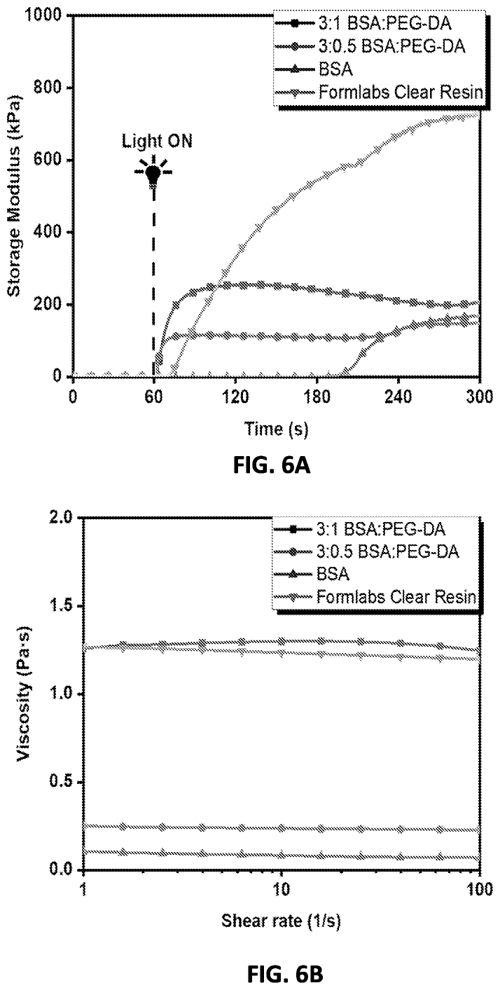

In this example, representative composition embodiments were prepared and evaluated for printability. The compositions were prepared as aqueous compositions comprising 30 wt % BSA, 10 wt % (or 5 wt %) PEG-DA, 0.24 wt % SPS, 0.075 wt % Ru(bpy)₃Cl, and 59.685 wt % DI water. The compositions were characterized by rheometry to determine the (i) the rate of photocure, and (ii) viscosity for each of the composition investigated. For comparison, the rheological measurements of a commercial SLA resin (Formlabs clear resin) was evaluated, along with a composition comprising the protein without any PEG-DA. Results for the photocure rate are shown in FIG. 6A. Results for viscosity measurements are shown in FIG. 6B.

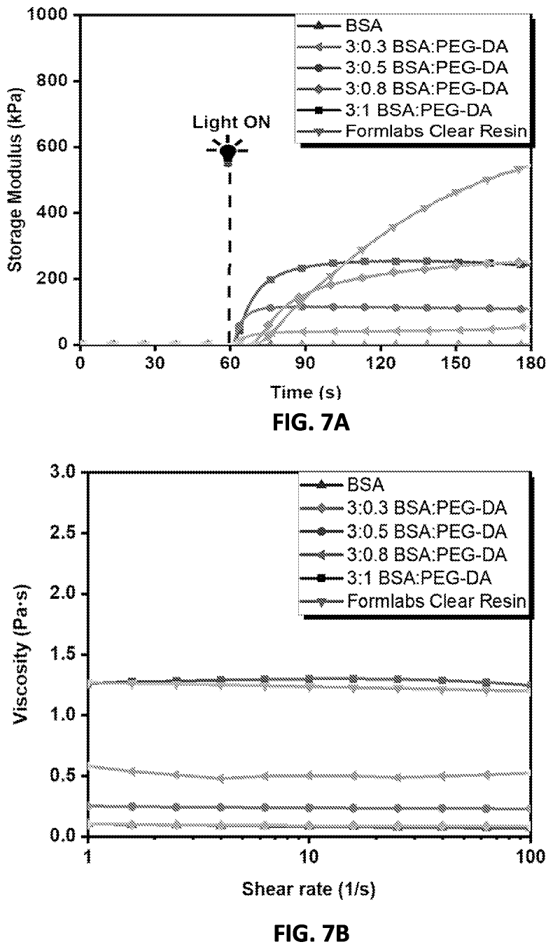

Additional results are shown for other embodiments in FIGS. 7A and 7B. FIG. 7A shows photo-rheology of a Formlabs clear commercial resin, 30 wt % BSA in DI water with 0.24 wt % SPS and 0.075 wt % Ru(bpy)₃Cl, and 30 wt % BSA with 0.24 wt % SPS and 0.075 wt % Ru(bpy)₃Cl and (i) 3 wt % PEG-DA, (ii) 5 wt % PEG-DA, (iii) 8 wt % PEG-DA, and (iv) 10 wt % PEG-DA. All the BSA-based formulations were prepared in DI water, and as last step, the photoinitiating system (0.24 wt % SPS and 0.075 wt % Ru(bpy)₃Cl) was added. A light source (365 nm) was turned on after 30 or 60 seconds, approximately the time to cure a layer in a Form 2 SLA printer. FIG. 7B shows viscosity vs shear rate data for the commercial resin, 30 wt % BSA, and 30 wt % BSA compositions with various amounts of co-monomer. The storage modulus (G′) was 3 kPa/s or more during the first seconds of UV light irradiation.

Table 1 provides rheometrical data for BSA composition formulations evaluated in this example. In some embodiments, two parameters for a printable composition using a Form 2 stereolithographic commercial printer are used to assess compositions, including compositions having a viscosity lower than 10 Pa·s, and a photo-curing rate based on the storage modulus (G′) during the first seconds of UV light irradiation higher than 3 kPa/s.

TABLE 1

| wt % | wt % | | Viscosity | G′ rate of change^{a} |
|---|---|---|---|---|
| BSA | Co-monomer | Printable | (Pa · s) | (kPa/s) |
| 30 | — | NO | 0.08 | 0 |
| 30 | 1 wt % PEG-DA | NO | 0.1 | 1.3 |
| 30 | 3 wt % PEG-DA | NO | 0.12 | 1.4 |
| 30 | 5 wt % PEG-DA | YES | 0.22 | 3.8 |
| 30 | 7.5 wt % PEG-DA | YES | 0.23 | 5.1 |
| 30 | 10 wt % PEG-DA | YES | 1.3 | 7.8 |
| 30 | 10 wt % PEG-A | NO | — | — |
| 30 | 10 wt % PEG-DMA | NO | — | — |

| | | | |
|---|---|---|---|
| Formlabs Clear Resin | YES | 1.2 | 4.8 |

^{a}Based on the first 30 seconds of irradiation.

### Example 2

FIGS. 5A and 5B show results from this example, where primary amine reaction with acrylate groups of a diacrylate-containing compound (e.g., PEG-DA) was assessed. FIG. 5A shows that the functionalization was demonstrated using a 2,4,6-trinitrobenzene sulfonate (TNBS) assay. Primary amines react with TNBS to form a compound that absorbs strongly at 342 nm. It was prepared from a composition composed of 30 wt % of BSA, 10 wt % of PEG-DA and 60 wt % of DI water, and after 3 days of dialysis, it was freeze dried to obtain a 3:1 BSA:PEG-DA powder. BSA and 3:1 BSA:PEG-DA were each dissolved in CB buffer at a concentration of 20 μg/mL. 0.25 mL of 0.01% (w/v) solution of TNBS was added to 0.5 mL of each protein solution. The samples were incubated at 37° C. for 2 hours. To quench the reaction, 0.25 mL of 10% SDS and 0.125 mL of 1 N HCl were added to each sample. The absorbance of each solution was measured at 342 nm. FIG. 5B shows the appearance of a peak at 1540 cm-1 in the ATR-FTIR spectrum of the 3:1 BSA:PEG-DA formulation, corresponds to the secondary amines formed when the lysine residues on the surface of BSA react with the acrylates of PEG-DA via an aza-Michael addition.

### Example 3

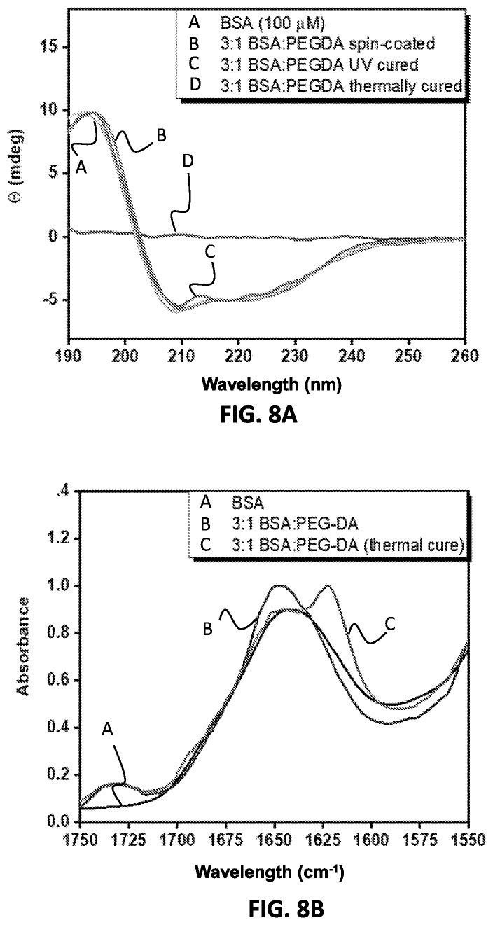

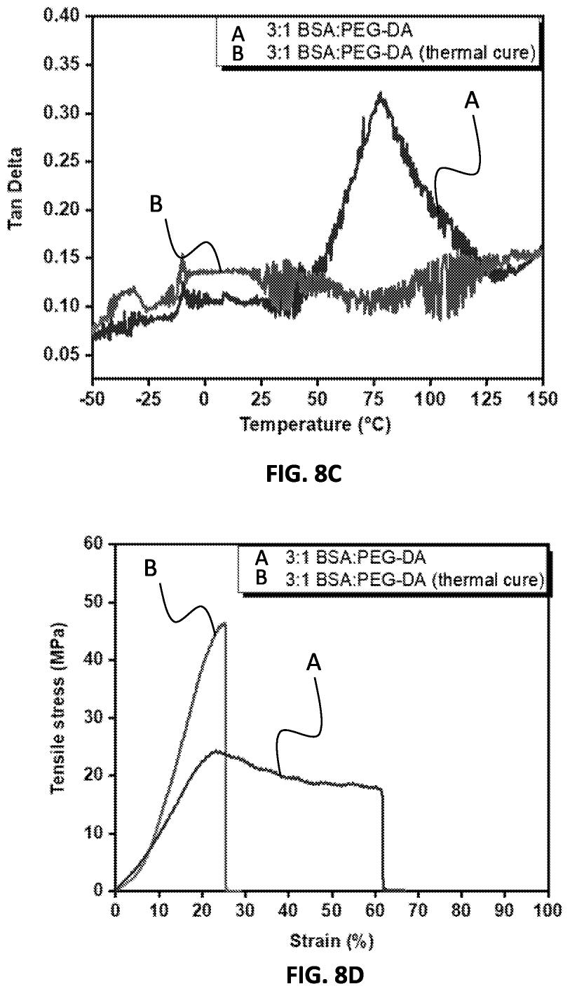

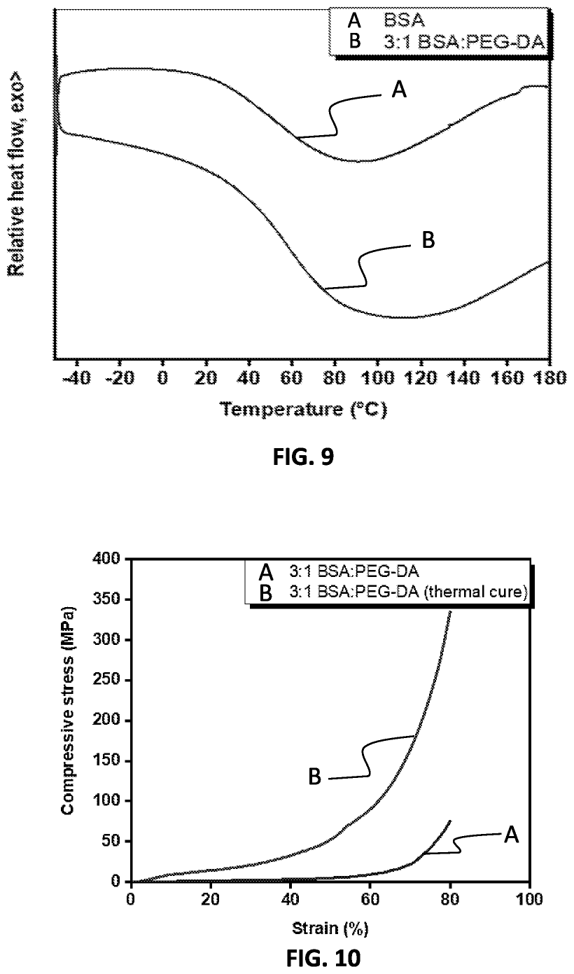

In this example, circular dichroism (CD) was used to evaluate the BSA protein conformation in the composition and printed objects. As demonstrated by CD spectroscopy, the BSA proteins maintain their conformation—both in the aqueous composition and the photocured bioplastic. See FIG. 8A, which shows solid state circular dichroism spectra showing that the BSA maintained its conformation after photocuring, but that this structure was lost after thermal cure (at 120° C.). The spectrum of the photocured composition after dehydration was characteristic of BSA in its folded conformation with two negative peaks around 210 and 222 nm, as well as the positive peak around 195 nm that reflected the high alpha-helical content of the protein. BSA is known to denature in solution at temperatures above 90° C., temperature that was also confirmed in dried bioplastics using differential scanning calorimetry (DSC). In some embodiments, both the BSA in the native state and the BSA-based bioplastic exhibited a denaturation process at a temperature above 90° C. See FIG. 9. The transition temperature was determined using DMTA, due to a superior sensitivity compared to the differential scanning calorimetry (DSC) in materials where the glass transition is so broad. The unfolding process of the proteins is an endothermic event that requires the input of energy, such as heat or force, to endow the proteins with internal mobility. Thermal treatment of 3D printed and dehydrated bioplastics (at 120° C.) caused the unfolding of the native BSA structure, which was reflected by the featureless CD spectrum (see FIG. 8A). This change in the secondary structure of the protein was also supported by the ATR-FTIR spectra in FIG. 8B, which shows ATR-FTIR spectra of the amide region shows the transition of the α-helical structure of BSA into β-sheets after thermal cure. In some embodiments, the amide I region of the spectrum showed a band at 1648 cm⁻¹, which was indicative of α-helical conformations, which decreased in favor of a new resonance at 1622 cm⁻¹ for the β-sheet interactions after thermal cure. The absence of thermal transitions observed after the heat treatment of the samples in the dynamic mechanical thermal analysis (DMTA) corroborates the unfolding of the native BSA. For example, see FIG. 8C, which shows a DMTA spectrum showed a glass transition at 78° C.

### Example 4

In this example, elastic modulus and strength of a thermally cured bioplastic object was evaluated, along with shape recovery properties. The β-sheet formation during thermal cure enhanced the stiffness of the materials, as demonstrated by the increase in the elastic modulus and strength of the thermally cured bioplastic. For example, see FIG. 8D. In some embodiments, the compressive elastic modulus varies from 10.5 MPa for non-heated samples to 103.5 to heated samples. Likewise, the compressive strength at the 80% of deformation raises from 75.5 MPa for the dehydrated samples to 334.9 MPa for thermally cured samples, as evidenced by the results in FIG. 10. Results are also summarized in Table 2, below.

TABLE 2

| Compression | Thickness | Diameter | Compressive |
|---|---|---|---|
| Cycle Number | (mm) | (mm) | Modulus (MPa)^{a} |
| 1 | 4.4 | 7.93 | 83.07 |
| 2 | 4.3 | 7.93 | 101.69 |
| 3 | 4.26 | 7.86 | 106.29 |
| 4 | 4.11 | 7.83 | 130.17 |
| 5 | 4.15 | 7.82 | 136.11 |
| 6 | 4.17 | 7.88 | 146.02 |
| 7 | 4.25 | 7.8 | 152.84 |
| 8 | 4.22 | 7.85 | 157.04 |
| 9 | 4.19 | 7.86 | 160.22 |
| 10 | 4.16 | 7.86 | 162.32 |

^{a}Compressive modulus was determined from the slope of the elastic region of the stress-strain curve

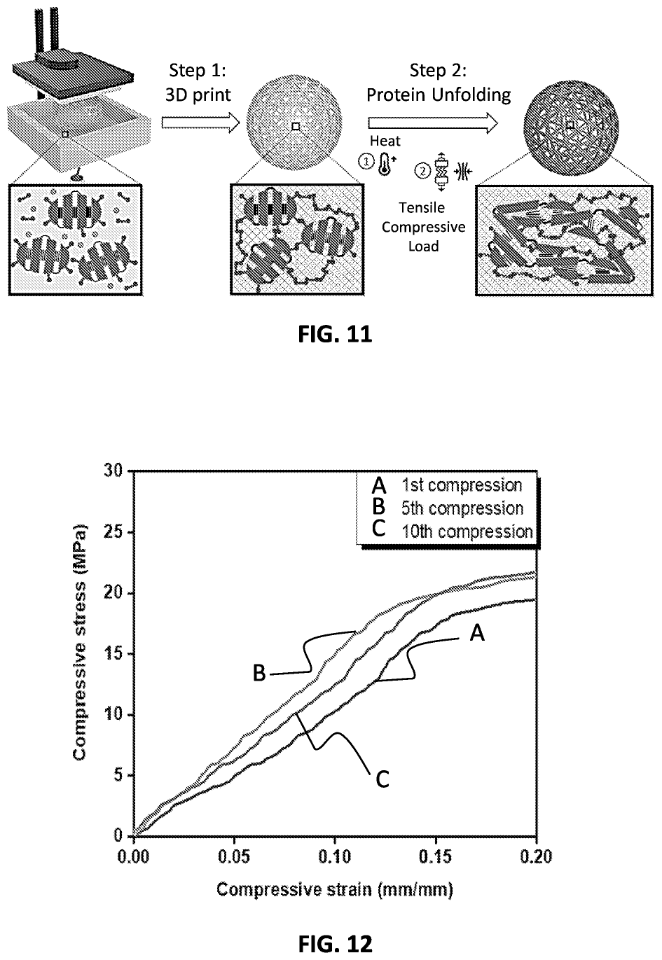

FIG. 8D shows representative uniaxial tensile stress-strain curve comparing thermally cured ((max=46 MPa) and non-thermally cured (σmax=24 MPa) samples; the ductility observed in the non-thermally cured sample is lost in the thermally cured sample as a result of the increased presence of intermolecular 3-sheets that were formed. This result was consistent with the presence of more intermolecular hydrogen bonding interactions between protein molecules. In some embodiments, the 3D printed bioplastic samples that were thermally cured to denature the globular proteins prior to mechanical analysis exhibited brittle fracture. The tensile strength was 46 MPa, while elongation at break was 25%. In contrast, the samples that were not thermally cured behaved as thermoplastic materials, with a clear yielding behavior upon stretching. The elongation at break increased to 61%, and the toughness (10.6 J·m⁻³) improved relative to the thermally cured and denatured samples (5.3 J·m⁻³). Thus, the folded globular proteins introduced a molecular plasticity, wherein the proteins were unfolded under tensile strain to release their stored length. This demonstration illustrates how the secondary structure of these 3D printed protein objects can be transformed from a thermoplastic to a thermoset using the same material composition. See the schematic illustration in FIG. 11, which shows a schematic depicting the unfolding of BSA as a consequence of either thermal treatment or mechanical load (tensile or compressive).

### Example 5

Based on the plasticity observed with these cross-linked thermosets (non-thermally cured), the shape-recovery of mechanically deformed 3D printed objects was evaluated. In another embodiment, a solid sphere was compressed to pass through a narrow opening. The 3D printed and dehydrated sphere with a diameter of 14 mm was too large to pass through an opening having dimensions of 20 mm×10 mm, so the sphere was compressed until it had a height of 9 mm (65% of the original height) so that it could slide through the opening. The spherical shape was recovered using a heat gun. In another embodiment, a printed “W” shaped rod with a length of 40 mm and a height of 12 mm could not fit through the opening of a plastic tube with diameter 8 mm, so it was manually stretched until it had a length of 55 mm and a height of 7 mm, such that it could fit within the tube.

The mechanically deformed objects maintained the deformed shape for indefinite periods of time. Each of these shapes recovered from their non-equilibrium shape when subjected to heating (˜120° C.) or rehydration in water. While the ductility of the 3D printed objects was derived from the release of the stored length in the globular proteins, the transition back to the respective equilibrium shapes was driven the presence of the covalently cross-linked network that was produced during the photo-initiated polymerization.

At high compressive strains, mechanical damage was observed in some embodiments of the bioplastic materials, but shape recovery was still achieved. However, when a compressive load of 20% was applied, there was no visible evidence of mechanical damage, and the 3D printed object could undergo over ten cycles of compression and thermal shape recovery without significant changes to the measured mechanical properties. FIG. 12 shows results from this example where compression & thermo-recovery cycles were evaluated. After 10 cycles of compression and recovery of the samples using heat, the same sample retains the initial compressive modulus that it has at the first compression.

### Example 6

A 3D printed husky dog was placed on top of four-dollar quarters, and these, in turn, were placed on the top of a cylindrical puck that was 3D printed and compressed to 50% of its initial height. This group of components was placed on the top of a heating plate at a temperature of 120° C., and the cylindrical puck was able to recover its shape prior to being compressed, as well as lift an overall weight of 25 grams in roughly 3 minutes. FIGS. 3A and 3B also show that the bioplastic obtained from the hydrogel printed form retained the same geometries and shapes as the printed hydrogel but with 30% smaller dimensions after drying.

### Example 7

A 3D printed hollow spherical lattice was compressed to 50% of its original diameter. Then, this compressed shape was submerged in water, and upon hydration, the spherical lattice regained its printed architecture. The complete recover of the original hydrogel occurred after 30 minutes of hydration. This example is shown in the bottom images of FIG. 1.

### Example 8

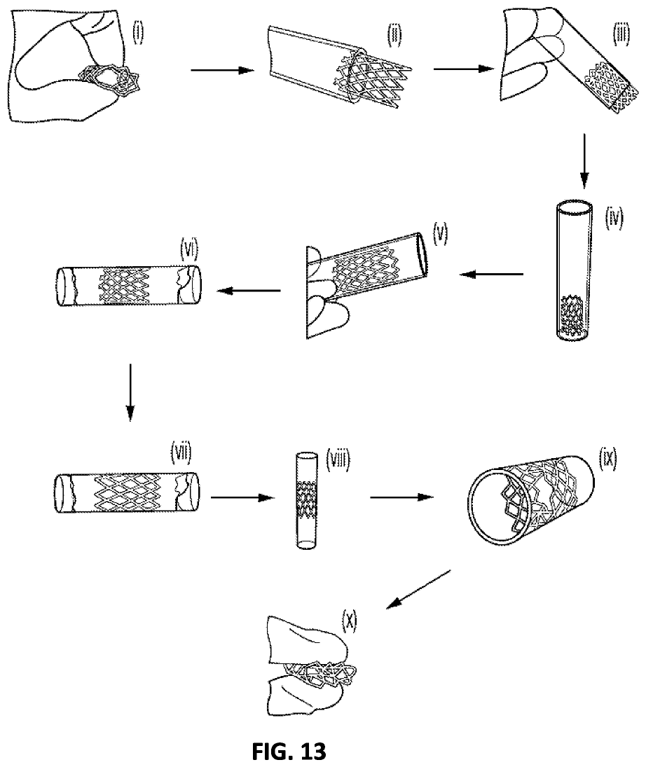

In this example, a printed stent was made using 30 wt % BSA, 10 wt % PEG-DA (700 g/mol), 0.24 wt % SPS, 0.075 wt % Ru(bpy)₃Cl, and 59.685 wt % DI water, and its properties were evaluated. Results are shown in FIG. 13, which provides illustrates of the observed results. In illustration (i) of FIG. 13, the printed stent exhibits flexibility in its dried state. Illustration (ii) of FIG. 13 shows that the stent was able to be fitted freely into the tube (as shown in illustrations (iii), (iv), and (v)) due to its smaller external diameter of (15 mm) as compared to the tube diameter (20 mm). Illustration (vi) of FIG. 13 shows that the stent after the tube was filled with water and capped and illustration (vii) shows the filled tube after 15 minutes, at which point the stent was perfectly adapted to the interior of the glass tube due to the swelling, increasing its length from 24 mm to 32 mm (30% bigger dimensions after swelling). In illustration (viii) and (ix) of FIG. 13, the stent was firmly fixed inside the tube; however, as shown in illustration (x), the stent was capable of being soften after hydration compared to the dried form and thus could be easily removed from the tube.

In view of the many possible embodiments to which the principles of the present disclosure may be applied, it should be recognized that the illustrated embodiments are only preferred examples of and should not be taken as limiting the scope of the disclosure. Rather, the scope is defined by the following claims. We therefore claim as our invention all that comes within the scope and spirit of these claims.

1. A composition, comprising:
a non-acrylated globular protein;
a diacrylate-containing compound;
a photoinitiating component; and
a solvent.

2. The composition of claim 1, wherein the composition has a viscosity that ranges from greater than 0 Pa·s to less than 10 Pa·s.

3. The composition of claim 1, wherein the non-acrylated globular protein and the diacrylate-containing compound are present at a ratio of 1:9 to 9:1 (non-acrylated globular protein to diacrylate-containing compound).

4. The composition of claim 1, wherein the non-acrylated globular protein and the diacrylate-containing compound are present at a ratio of 1:3 to 3:1 (non-acrylated globular protein to diacrylate-containing compound).

5. The composition of claim 1, wherein the non-acrylated globular protein is an albumin protein, a pepsin protein, a hemoglobin protein, an enzyme, a lysozyme, or a combination thereof.

6. The composition of claim 1, wherein the diacrylate-containing compound is a diacrylate-containing poly(heteroaliphatic) polymer.

7. The composition of claim 1, wherein the diacrylate-containing compound is a poly(alkylene oxide)-diacrylate compound, a poly(alkylene amine)-diacrylate compound, a poly(alkylene thiol)-diacrylate compound, or a combination thereof.

8. The composition of claim 1, wherein the photoinitiating component comprises ruthenium trisbipyridine chloride, lithium phenyl-2,4,6-trimethylbenzoylphosphinate (LAP), 2-hydroxy-2-methylpropiophenone, benzophenone, ethyl 2,4,6-trimethylbenzoylphenyl phosphinate, 1-hydroxy-cyclohexyl-phenyl-ketone, 2-hydroxy-2-methyl-1-phenyl-1-propanone, 2,2-dimethoxy-2-phenyl acetophenone, 2-hydroxy-1-[4-(2-hydroxyethoxy)phenyl]-2-methyl-1-propanone, and diphenyl (2,4,6-trimethylbenzoyl)-phosphine oxide, sodium persulfate, ammonium persulfate, or any combination thereof.

9. The composition of claim 1, wherein the solvent is water.

10. The composition of claim 1, wherein the photoinitiating component is present in an amount between 0 wt % and 20 wt % of the diacrylate-containing compound.

11. The composition of claim 1, wherein the non-acrylated globular protein is covalently bound to the diacrylate-containing compound through a covalent bond formed between an amine group of the non-acrylated globular protein and a carbon atom of the diacrylate-containing compound.

12. The composition of claim 1, wherein:
the non-acrylated globular protein is non-acrylated bovine serum albumin;
the diacrylate-containing compound is a diacrylate-containing poly(ethylene glycol) compound;
the photoinitiating component comprises sodium persulfate and ruthenium tris(bipyridyl) chloride; and
the solvent is water.

13. A printed object, comprising a polymerized network comprising a first globular protein molecule that is covalently bound to a saturated form of a first diacrylate-containing compound, wherein the protein is directly covalently bound to a carbon atom of the saturated form of the first diacrylate-containing compound via a non-acrylated functional group of the protein; and wherein the saturated form of the diacrylate-containing compound is further directly covalently bound to a saturated form of a second diacrylate-containing compound, a second globular protein molecule, or a combination thereof.

14. The printed object of claim 13, wherein the first globular protein molecule is a bovine serum albumin molecule that is covalently bound to a saturated form of a first PEG-diacrylate compound such that the bovine serum albumin molecule is directly covalently bound to a carbon atom of the saturated form of the first PEG-diacrylate compound via an amine group of the bovine serum albumin molecule; and wherein the saturated form of the first PEG-diacrylate compound is further directly covalently bound to a saturated form of a second PEG-diacrylate compound, a second bovine serum albumin molecule, or a combination thereof.

15. The printed object of claim 13, wherein the printed object exhibits shape recovery behavior.

16. A method, comprising printing an object using a composition comprising (i) a non-acrylated globular protein; (ii) a diacrylate-containing compound; (iii) a photoinitiating component; and (iv) a solvent, wherein the printing is carried out using an additive manufacturing device that uses an energy source to promote vat photopolymerization.

17. The method of claim 16, further comprising drying the object.

18. The method of claim 16, wherein the additive manufacturing device is a printer capable of stereolithography, digital light processing, continuous liquid interface production, high-area rapid printing, daylight polymer printing, or a combination thereof.

19. The method of claim 16, further comprising (i) exposing the object to heat; (ii) hydrating the object; (iii) deforming the object; or (iv) a combination of (i), (ii), and/or (iii).

20. The method of claim 19, wherein the method comprises exposing the object to heat after the object is printed and after any drying step, and then deforming the object after it has been exposed to the heat.
